(19) United States
(12) Patent Application Publication
(10) Pub. No.: US 2023/0037534 A1
(43) Pub. Date: Feb. 9, 2023

(54) UTILIZATION OF A FASTING-MIMICKING DIET TO REDUCE SENESCENT CELLS AND INFLAMMATION

(71) Applicant: UNIVERSITY OF SOUTHERN CALIFORNIA, Los Angeles, CA (US)

(72) Inventors: Valter D. LONGO, Los Angeles, CA (US); Sebastian BRANDHORST, Los Angeles, CA (US); Novella GUIDI, Los Angeles, CA (US); Annunziata Nancy CRUPI, Los Angeles, CA (US)

(73) Assignee: UNIVERSITY OF SOUTHERN CALIFORNIA, Los Angeles, CA (US)

(21) Appl. No.: 17/786,481

(22) PCT Filed: Dec. 16, 2020
(86) PCT No.: PCT/US2020/065295

§ 371 Date: Jun. 16, 2022

Related U.S. Application Data
(60) Provisional application No. 62/948,434, filed on Dec. 16, 2019.

(51) Int. Cl. A23L 33/00; G01N 33/68; A23L 33/125; A23L 33/115; A23L 33/17

(52) U.S. Cl. CPC A23L 33/30; G01N 33/6893; A23L 33/125; A23L 33/115; A23L 33/17; G01N 2800/7042

(57) ABSTRACT

A method for reducing senescence-positive cells in a subject includes a step of identify a subject having increased expression of p16INK4a or at risk for increased of p16INK4a. A fasting-mimicking diet is administered to the subject for a first time period.

### CROSS-REFERENCE TO RELATED APPLICATIONS

This application claims the benefit of U.S. provisional application Ser. No. 62/948,434 filed Dec. 16, 2019, the disclosure of which is hereby incorporated in its(their) entirety by reference herein.

### TECHNICAL FIELD

In at least one aspect, the present invention is related to dietary methods for reducing senescence-positive cells.

### BACKGROUND

Cellular senescence, a stress-inducible irreversible growth arrest, plays a pivotal role in preventing the proliferation of preneoplastic cells. However, with increasing age, senescent cells accumulate, avoid apoptosis, prevail for prolonged periods in a cell-cycle arrested state, and are thought to deplete stem- and progenitor cells. Cellular senescence is characterized by the expression of p16INK4a and a distinct senescence-associated secretory phenotype.

Prolonged fasting and a periodic low-protein, low-calorie ketogenic fasting-mimicking diet (FMD) promote stress resistance, delay aging and disease progression and induce cellular atrophy in multiple systems and organs that are followed by the regeneration of various cell types upon refeeding (1, 2).

It has previously been demonstrated that the FMD improves lifespan and muscle performance in old mice (2). In a clinical trial, three FMD cycles decreased risk factors/biomarkers for aging, diabetes, cardiovascular disease without major adverse effects, providing support for the use of FMDs to improve healthspan (1).

The targeted removal of p16INK4-positive cells, a cell senescence marker, extends the median lifespan, delays the onset of tumorigenesis, attenuates the age-related deterioration of tissue homeostasis and function, reverses the myeloid bias, and rejuvenates hematopoietic stem cells in aged mice. Notably, these effects largely overlap with fasting-induced benefits on health and lifespan.

Senescent cells are characterized by several changes, including the enhanced secretion of proinflammatory molecules. Chronic inflammation is among the most significant consequences of senescence in aging tissue. Increasing evidence shows that C-reactive protein (CRP) is an inflammatory biomarker but also an important risk factor associated with aging-related diseases. Studies have shown that levels of CRP, an acute-phase protein, are increased in aging-related disease(s). Systemic inflammation is reflected by an increase in white blood cell and lymphocyte numbers. Reducing senescent cells, thus also reduces inflammation.

As of today, no dietary intervention has been utilized to induce specific senolytic effects in mammalian aging.

Accordingly, there is a need for improved methods for reducing senescence-positive cells.

### SUMMARY

In at least one aspect, the present invention provides a novel dietary approach to induce health and lifespan benefits using prolonged fasting and a ketogenic fasting-mimicking diet. This approach functions as a senolytic intervention that targets senescent cells.

In another aspect, a method for reducing senescence-positive cells is provided. The method includes steps of identifying a subject having increased expression of p16INK4a or at risk for increased of p16INK4a and administering (or providing) a fasting-mimicking diet (FMD) to the subject for a first time period after the identification step.

In another aspect, a method for reducing senescence-positive cells is provided. The method includes a step of administering (or providing) a fasting-mimicking diet (FMD) to a subject for a first time period, the subject having been identified at increased expression of p16INK4a or at risk for increased of p16INK4a.

In another aspect, a method for prophylactically protecting against senescence-positive cells is provided. The method includes a step of administering (or providing) a fasting-mimicking diet (FMD) to a subject for a first time period.

Prolonged fasting and the ketogenic fasting-mimicking diet can be considered feasible in a wide range of subjects and with no collateral effects. Advantageously, these interventions could specifically remove senescent cells.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1A. p16 expression as a function of p16(luc) mice age.

FIG. 1B. p16 expression in p16(luc) mice fast a first FMD cycle, a refeeding period, and a second FMD cycle.

FIG. 1C. Percent LT-HSC cas3+ apoptotic cells after a FMD cycle and 24 hour refeeding.

FIG. 1D. Percent p16⁺ LT-HSC cas3+ apoptotic cells after a FMD cycle and 24 hour refeeding.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within ±0%, 0.1%, 0.5%, 1%, 2%, 3%, 4%, 5% or 10% of the value or relative characteristic.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5. 1.6, 1.7, 1.8, 1.9, and 2.0 can be selected as lower or upper limits. In the specific examples set forth herein, conditions can be practiced with plus or minus 50 percent of the values indicated rounded to three significant figures. In a refinement, conditions can be practiced with plus or minus 30 percent of the values indicated rounded to three significant figures of the value provided i.n the examples. In another refinement, conditions can be practiced with plus or minus 10 percent of the values indicated rounded to three significant figures of the value provided in the examples.

Throughout this application, where publications are referenced, the disclosures of these publications in their entireties are hereby incorporated by reference into this application to more fully describe the state of the art to which this invention pertains.

Abbreviations:

“FMD” means ketogenic fasting-mimicking diet.

“LT-HSCs” means long-term hematopoietic stem cells.

“MPPs” means multipotent progenitors.

“p/s” means photons per second.

“RDA” means recommended dietary allowance.

“RF” means refeeding.

“RGAS” means Generally Regarded As Safe.

“ST-HSCs” means short-term hematopoietic stem cells.

“p16INK4a” is a tumor suppressor and aging biomarker. Sometimes p16INK4a is referred to as p16^{INK4a}, p16, p16(INK4a), cyclin-dependent kinase inhibitor 2A, CDKN2A, multiple tumor suppressor 1, and other synonyms known in the art.

“p16(LUC)” is a luciferase knock-in mouse for p16.

“STS” means short-term starvation.

“WBC” means White Blood Cells.

“CRP” means C-reactive protein.

The terms “kilocalorie” (kcal) and “Calorie” refer to the food calorie. The term “calorie” refers to the so-called small calorie.

The term “senolytic” means a treatment that selectively induces death of senescent cells.

“Short-term starvation” means no caloric intake for a given day. In a refinement, “short-term starvation” means less than 100 kcal. of caloric intake in a given day. In another refinement, “short-term starvation” means less than 200 kcal. of caloric intake in a given day.

The term “subject” refers to a human or animal, including all mammals such as primates (particularly higher primates), sheep, dog, rodents (e g., mouse or rat), guinea pig, goat, pig, cat, rabbit, and cow.

The term “fasting-mimicking diet” (FMD) means a ketogenic diet that mimics the effects of fasting typically by providing a subject with at most 50% of their normal caloric intake but with some nutritional component so that fasting is mimicked while a subject is not completely starved. Therefore, the fasting-mimicking diet provides the health benefits of fasting while still nourishing the subject. Since the subjection is provided nourishment, compliance is better than pure fasting. Examples of useful fasting-mimicking and enhancing diets and methods for monitoring the effects of these diets on markers such as IGF-1 and IGFBP1 in the context of the present invention are set forth in U.S. patent application Ser. No. 14/273,946 filed May 9, 2014; Ser. No. 14/497,752 filed Sep. 26, 2014; Ser. No. 12/910,508 filed Oct. 22, 2010; Ser. No. 13/643,673 filed Oct. 26, 2012; Ser. No. 13/982,307 tiled Jul. 29, 2013; Ser. No, 14/060,494 filed Oct. 22, 2013; Ser. No. 14/178,953 filed Feb. 12, 2014; Ser. No. 14/320,996 filed Jul. 1, 2014; Ser. No. 14/671,622 filed Mar. 27, 2015; the entire disclosure of these patent applications is hereby incorporated by reference. The lasting-mimicking diet set forth in U.S. patent application Ser. Nos. 14/060,494 and 14/178,953 are found to be particularly useful in the present invention. Additional examples of FMD diets are found in U.S. patent application Ser. No. 15/148,251 and WIPO Pub. No. WO2011/050302 and WIPO Pub. No. WO2011/050302; the entire disclosures of which are hereby incorporated by reference.

In an embodiment, a method for reducing senescence-positive cells in a subject is provided. The method includes a step of identifying a subject having increased expression of p16INK4a or at risk for increased expression of p16INK4a. In this context, increased expression means a level of expression for p16 that is higher than a predetermined baseline. In a refinement, the mere detection of p16INK4a can be sufficient for identifying a subject at risk and potentially benefiting from the method for reducing senescence-positive cells. In another refinement, immunohistochemistry can be used to detect the presence of the p16INK4a biomarker. (see for example, Nielsen G P, Stemmer-Rachamimov A O, Shaw J, Roy J E, Koh J, Louis D N. Immunohistochemical survey of p16INK4A expression in normal human adult and infant tissues. Lab Invest. 1999; 79:1137-43); the entire disclosure of which is hereby incorporated by reference. In other variations, increased expression of p16 or the risk thereof is inferred by identifying subjects at risk for cancer or a subject being older than a predetermined age. Such risks for cancer include, but are not limited to, diabetes, family history, obesity, advanced age (e.g., over the predetermined age), smoking, exposure to cancer-causing chemicals, exposure to radiation, and the like. Typically, the predetermined age is from 45 to 70 years. The predetermined age is, in order of increasing preference, 45 years, 50 years, 60 years, or 70 years. In this embodiment and all refinements, a fasting-mimicking diet (FMD) as set forth herein is provided to or administered to the subject for a first time period (e.g., after the identification step). In some variations, a second diet is administered to (or provided to) the subject for a second time period as set forth below.

In another embodiment, a method for reducing white blood cell counts or lymphocyte counts in subjects with elevated systemic inflammation, as indicated by increased CRP levels. The method includes a step of identifying a subject having high levels of CRP at baseline. In this context, increased expression means a level of expression for CRP that is higher than 1 mg/L predetermined baseline. In some refinements, increased expression means a level of expression for CRP that is higher than 0.1 mg/L, 0.5 mg/L, 1 mg/L, 2 mg/L, or 3 predetermined baseline. The predetermined baseline can be determine as the average of a population of healthy individuals. In this context, healthy individuals are subjects not having a diagnosed cancer and/or do not have risk factors (diabetes, family history, obesity, advanced age (e.g., over 65 years), smoking, exposure to cancer-causing chemicals, exposure to radiation and the like.) In a refinement, the mere detection of CRP can be sufficient for identifying a subject at risk and potentially benefitting from the method for reducing GRP levels. In another refinement, immunohistochemistry can be used to detect the presence of CRP. Reduced systemic inflammation can be identified by measuring a reduction in WBC and/or lymphocyte levels.

In another embodiment, a method for prophylactically protecting against senescence-positive cells is provided. The method includes a step of administering (or providing) a fasting-mimicking diet (FMD) to a subject for a first time period. In some variations, a second diet is administered to (or provided to) the subject for a second time period as set forth below.

After a subject has been identified in each of the embodiments, variations, and refinements set forth herein, an FMD is then administered to (or provided) the identified subject for a first time period. The administration (or providing) of the FMD for a time period of consecutive days is an FMD cycle. In a refinement, the first time period is 1 to 10 days. In a further refinement, the first time period is 1, 2, 3, 4, or 5 days. In a variation, the subject fasts for the first time period. In this regard, prolonged fasting describes a no-calorie to very-low-calorie intervention lasting more than at least one full day.

In some variations, a second diet is administered to (or provided to) the subject for a second time period. The second diet provides an overall calorie consumption that is within 10 percent of a subject's normal calorie consumption. In this context, normal calorie consumption is the number of calories needed to maintain the subject's weight at their pretreatment weight or to maintain a predetermined target weight. Therefore, the second diet can be referred to as a “refeeding diet.” Although the present invention is not significantly limited by the second time period, the second time period can be from 7 days to 6 months or longer. Typically, the second time period is from 10 days to 26 days or longer following the fasting-mimicking and enhancing diet. In some refinements, the second diet provides at most, in increasing order of preference, 2500 kcal/day, 2400 kcal/day, 2300 kcal/day, 2200 kcal/day, 2100 kcal/day, 2000 kcal/day, 1900 kcal/day, 1800 kcal/day, 1700 kcal/day, 1600 kcal/day, or 1500 kcal/day. In some further refinements, the second diet provides at least, in increasing order of preference, 1200 kcal/day, 1300 kcal/day, 1400 kcal/day, 1500 kcal/day, 1600 kcal/day, 1700 kcal/day, or 1800 kcal/day.

In a variation, STS or a FMD is repeated a plurality of times at predetermined intervals. In a further refinement, STS or a FMD is administered (or provided) for the first time period (e.g., 5 days) every 2-24 weeks or 1-2 days every week. For example, STS or a FMD can be repeated at intervals from one week to 6 months. Typically, the subject is administered (or provided) a normal diet (e.g., the refeeding diet) in between these repetitions.

In one variation, the FMD is a modified human FMD that includes ingredients, which are Generally Regarded As Safe (RGAS). The FMD includes specific requirements for day 1 of the FMD and for any additional days of the diet. Typically, the FMD includes 2 to 10 additional days. In a refinement, the FMD includes 2 to 4 additional days. In a further refinement, the FMD includes 4 additional days for a total of 5 days for the FMD cycle. The FM D includes lyophilized vegetable soups, bars, olives, crackers, herbal teas, supplements of vitamins and minerals, available for purchase in the form of 5-days diet boxes, containing everything necessary for 5 days of diet. For day 1, total calorie consumption is 3.5-5 kcal per pound of the subject's weight (or 7.7-11 kcal per kilogram). The daily diet should contain less than 25 g of sugar, less than 23 g of proteins, 16-25 grams of monounsaturated fats, 4.8-8 g of polyunsaturated fats and 1-10 g of saturated fats. For additional days of the FMD (e.g., 2 to 10 additional days (typically days 2-4), total calorie consumption is 2.4-4 kcal per pound (or 5.3-8.8 kcal per kilogram). The diet should contain less than 16 g of sugars, less than 15 g of protein, 8-12 g of monounsaturated fats, 2-4 g of polyunsaturated fats and 1-6 grams of saturated fats. Diet should also be high nourishment containing 100% of the RDA for vitamins, minerals and essential fatty acids. In a variation, for day 1, the FMD daily diet should include sugar in an amount of at least, increasing order of preference, 10 g, 8 g, 6 g, 4 g, 2 g, or 1 g of sugar and at most, 25 g, 22 g, 20 g, 18 g, 15 g, or 12 g and protein in an amount of at least, in increasing order of preference, 10 g, 8 g, 6 g, 4 g, 2 g, or 1 g and at most, in increasing order of preference, 25 g, 23 g, 20 g, 18 g, 15 g, or 12 g. For any additional days (typically days 2-4), the FMD daily diet should include sugar in an amount of at least, in increasing order of preference, 5 g, 4 g, 3 g, 2 g, 1 g, or 0.5 g of sugar and at most, 20 g, 18 g, 16 g, 14 g, 12 g, or 10 g and protein in an amount of at least, in increasing order of preference, 5 g, 4 g, 3 g, 2 g, 1 g, or 0.5 and at most, in increasing order of preference, 20 g, 18 g, 15 g, 12 g, 10 g, or 10 g

In a refinement, a 1 to 10 day (typically a 5 day) supply of the FMD includes one or more or all or the following components: soups/broths, soft drinks, nut bars, and supplements.

In another variation, the FMD is a low protein diet. Characteristically, the low protein diet provides percent calories from protein that is less than a predetermined cutoff protein intake level. Typically, the predetermined cutoff protein intake level is 20% calories from proteins of the total calories consumed on average per day by the subject. In a refinement, the predetermined cutoff protein intake level is 15% calories from proteins of the total calories consumed on average per day by the subject. In a refinement, the predetermined cutoff protein intake level is 10% calories from proteins of the total calories consumed on average per day by the subject. In another refinement, the predetermined cutoff protein intake level is 5% calories from proteins of the total calories consumed on average per day by the subject. In some refinements, the low protein diet provides greater than, in increasing order of preference, 40%, 50%, 60%, 70%, 80%, and 90% calories from protein that are plant sources such as soybeans. (note, overall, protein is still less than 20% of the total calories that are consumed. Advantageously, the low protein diet provides about 100% calories of protein from plant sources.

In a variation, the FMD includes the following micronutrients (at least 95% non-animal based): over 5,000 IU of vitamin A per day (days 1-5); 60-240 mg of vitamin C per day (days 1-5); 400-800 mg of Calcium per day (days 1-5); 7.2-14.4 mg of Iron per day (days 1-5); 200-400 mg of Magnesium per day (days 1-5); 1-2 mg of copper per day (days 1-5); 1-2 mg of Manganese per day (days 1-5); 3.5-7 mcg of Selenium per day (days 1-5); 2-4 mg of Vitamin B1 per day (days 1-5); 2-4 mg of Vitamin B2 per day (days 1-5); 20-30 mg of Vitamin B3 per day (days 1-5); 1-1.5 mg of Vitamin B5 per day (days 1-5); 2-4 mg of Vitamin B6 per day (days 1-5); 240-480 mcg of Vitamin B9 per day (days 1-5); 600-1000 IU of Vitamin D per day (days 1-5); 14-30 mg of Vitamin E per day (days 1-5); over 80 mcg of Vitamin K per day (days 1-5); 16-25 mcg Vitamin B12 are provided during the entire 5-day period; 600 mg of Docosahexaenoic acid (DHA, algae-derived) are provided during the entire 5-day period. The FMD diet provides high micronutrient content mostly (i.e., greater than 50 percent by weight) from natural sources including: Kale, Cashews, Yellow Bell Pepper, Onion, Lemon Juice, Yeast, Turmeric. Mushroom, Carrot, Olive Oil, Beet Juice, Spinach, Tomato, Collard, Nettle, Thyme, Salt, Pepper, Vitamin B12 (Cyanocobalamin), Beets, Butternut Squash, Collard, Tomato, Oregano, Tomato Juice, Orange Juice, Celery, Romaine Lettuce, Spinach, Cumin, Orange Rind, Citric Acid, Nutmeg, Cloves, and combinations thereof.

In another variation, the FMD diet includes over 50% of the recommended daily value of dietary fiber on all days. In a refinement, the amount of dietary fiber is greater than 15 grams per day on all days (e.g., 5 days). In a further refinement, the FMD includes 12-25 grams of glycerol per day on days 1 and/or any additional days (e.g. days 2-5). In a refinement, glycerol is provided at 0.1 grams per pound body weight/day.

Although the ketogenic FMDs encompasses virtually any source of fat, sources high in unsaturated fat, including monounsaturated and polyunsaturated fat sources, are particularly useful (e.g., omega-3/6 essential fatty acids). Suitable examples of monounsaturated food sources include, but are not limited to, coconut, coconut oil, olive oil, flaxseed, peanut butter, olives, nuts (e.g., almonds, macadamia nuts, pecans, pistachios, cashews), avocado, seeds (e.g., sesame), oils (e.g., olive, sesame, peanut, canola), etc. Suitable examples of polyunsaturated food sources include, but are not limited to, walnuts, seeds (e.g., pumpkin, sunflower), flaxseed, fish (e.g., salmon, tuna, mackerel), oils (e.g., safflower, soybean, corn). The FMDs also includes a component selected from the group consisting of vegetable extracts, minerals, omega-3/6 essential fatty acids, and combinations thereof. In one refinement, such a vegetable extract provides the equivalent of 5 recommended daily servings of vegetables. Suitable sources for the vegetable extract include, but are not limited to, bok choy, kale, lettuce, asparagus, carrot, butternut squash, alfalfa, green peas, tomato, cabbage, cauliflower, beets. Suitable sources for the omega-3/6 essential fatty acids include fish such as salmon, tuna, mackerel, bluefish, swordfish, and the like. In a further refinement, the FMDs includes fat sources such that at least 25 percent of calories from fat are short-chain fatty acids having from 2 to 7 carbon atoms and/or from medium-chain saturated fatty acids having from 8 to 12 carbon atoms. Specific examples of fatty acids include lauric and/or myristic acid. In other refinements, the FMDs include calories from fat in an amount from about 0 to 22 percent of total calories contained in the diet.

In another embodiment, a diet package for implemented the FMD diet protocol set forth above is provided. A particularly useful example of such a diet package is set forth in US Pat Pub No. US20180228198A; the entire disclosure of which is hereby incorporated by reference. Characteristically, the diet package partitions the FMD components (and caloric amounts) into meal portions for each day of the FMD. In a further refinement, the diet package provides instructions providing details of the methods set forth above.

In other variations, the diet package and/or the FMD is used to induce or make a medicament for inducing health and lifespan benefits using prolonged fasting and a ketogenic fasting-mimicking diet. In still other variations, the diet package and/or the FMD is used to reduce (i.e., treat) or make a medicament to reduce senescence-positive cells as set forth above. In still other variations, the diet package and/or the FMD is used to prophylactically protect or make a medicament to prophylactically protect against senescence-positive cells (e.g., subjects having increased amounts of senescence-positive cells) as set forth above.

The following examples illustrate the various embodiments of the present invention. Those skilled in the art will recognize many variations that are within the spirit of the present invention and scope of the claims.

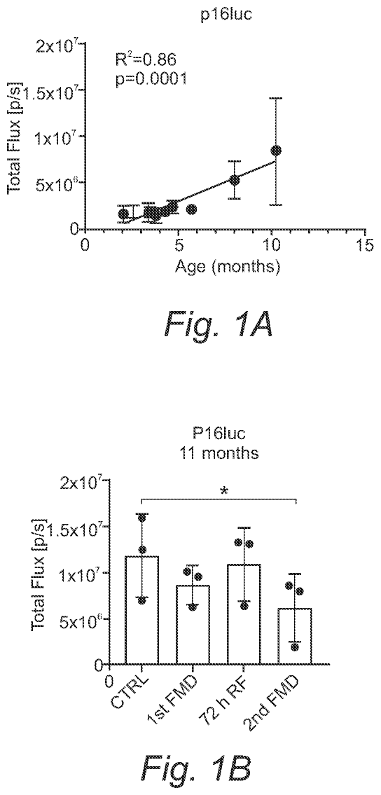

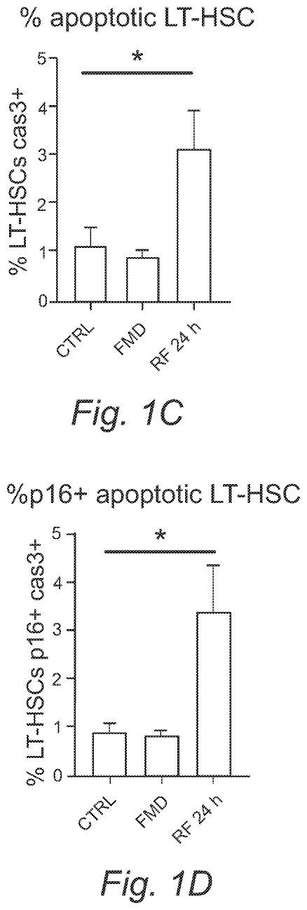

With reference to FIGS. 1A-1D, mice underwent 2 monthly cycles of FMD, while following a normal chow diet (CTRL) during the rest of the month. As a control, age and sex matched mice were maintained on a regular chow diet without exposure to the FMD. Similarly, prolonged fasting can be applied. Compared to their untreated baseline measurements, we noted reduced whole-body bioluminescence immediately after the 2nd FMD cycle. Thus, these results in the oldest mice indicate an increase in p16 related bioluminescence that was successfully reduced after 2 cycles of the FMD.

After 2 FMD cycles, the bone marrow of 22 months-old male and female control-fed and FMD-fed C57BL/6 mice immediately at the end of the FMD and 24 hours after refeeding was collected to measure p16 expression. Notably, p16 expression was reduced within the population of long-term hematopoietic stem cells (LT-HSCs), but not short-term hematopoietic stem cells (ST-HSCs) or multipotent progenitors (MPPs) 24 hours after refeeding. This reduction in p16 expression 24 hours after completion of the FMD was associated with apoptosis as shown by the predominant activation of caspase 3 within the population of old LT-HSC cells.

With reference to Table 1, the levels of WBCs and lymphocytes, and the effect of 3 cycles of a 5-day FMD on them in human subjects with elevated CRP (>1 mg/L) is demonstrated. In subjects with elevated CRP, the WBC counts are increased compared to those in subjects with CRP in the normal range (<1 mg/L). These were reduced at the end of FMD cycle 1, as well as after 3 FMD cycles. Lymphocyte numbers are also apparent at the end of FMD cycle 1 for the higher CRP group. These results suggest that FMD cycles can reduce senescence-associated systemic inflammation.

Table 1 provides White Blood Cell (WBC) and Lymphocyte counts from patients with low C-reactive protein (CRP; <1 mg/L; n=36) or higher CRP (>1 mg/L) at baseline. The FMD intervention reduced WBC and lymphocyte counts at the end of an initial 5-day FMD cycle before resuming normal food intake (b) (n=25), and reduced WBC approximately 5 days after completing 3 FMD cycles and refeeding (c) (n=25). * unpaired t-test between groups baseline CRP >1 mg/L vs. baseline CRP <1 mg/L. ^{#} paired t-test compared to baseline CRP >1 mg/L.

TABLE 1
White Blood Cell (WBC) and Lymphocyte counts from patients with low C-reactive protein

| | | | CRP > 1 mg/L | | | | | | | | |
|---|---|---|---|---|---|---|---|---|---|---|---|
| | | | | | | | | | Refeeding after | | |
| | CRP < mg 1 | | Baseline | | | End of 1^{st} FMD | | | 3^{rd} FMD | | |
| | Baseline | | | | ^{#}p- | | | ^{#}p- | | | ^{#}p- |
| | Mean ± SD | | Mean ± SD | | value | Mean ± SD | | value | Mean ± SD | | value |
| WBC | 5.06 | 1.41 | 5.93 | 1.42 | 0.021 | 5.67 | 1.38 | 0.039 | 5.32 | 1.40 | 0.025 |
| (10³/ml) | | | | | | | | | | | |
| Lymphocytes | 1.61 | 0.57 | 1.82 | 0.46 | 0.13 | 1.65 | 0.44 | 0.028 | 1.83 | 0.56 | 0.91 |
| (10³/ml) | | | | | | | | | | | |

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. Wei M, Brandhorst S, Shelehchi M, Mirzaei H, Cheng C W, Budniak J, Groshen S, Mack W J, Guen E, Di Biase S, Cohen P, Morgan T E, Dorff T, Hong K, Michalsen A, Laviano A, Longo V D, Fasting-mimicking diet and markers/risk factors for aging, diabetes, cancer, and cardiovascular disease. Sci Transl Med. 2017 Feb. 15; 9(377). pii: eaai8700

2. Brandhorst S, Choi I Y, Wei M, Cheng C W, Sedrakyan S, Navarrete G, Dubeau L, Yap L P, Park R, Vinciguerra M, Di Biase S, Mirzaei H, Mirisola M G, Childress P, Ji L, Groshen S, Penna F, Odetti P, Perin L, Conti P S, Ikeno Y, Kennedy B K, Cohen P, Morgan T E, Dorff T B, Longo V D. A Periodic Diet that Mimics Fasting Promotes Multi-System Regeneration, Enhanced Cognitive Performance, and Healthspan. Cell Metab. 2015 Jul. 7; 22(1):86-99

1. A method for reducing senescence-positive cells comprises:
a) identifying a subject having increased expression of p16INK4a or at risk for increased of p16INK4a; and
b) administering a fasting-mimicking diet (FMD) to the subject for a first time period after step a).

2. The method of claim 1, wherein the first time period is 1 to 10 days.

3. The method of claim 1, wherein the first time period is 5 days.

4. The method of claim 1, wherein a level of p16 expression higher than an average value for human subjects.

5. The method of claim 1, wherein the subject is identified in step a) by determining if the subject has a risk factor for cancer.

6. The method of claim 1, wherein the subject is identified in step a) by determining if the subject is older than a predetermined age.

7. The method of claim 1, wherein the subject is older than a predetermined age is 45 years.

8. The method of claim 1, wherein the FMD provides 3.5-5 kcal per pound of a subject's weight for day 1 of the FMD and 2.4-4 kcal per pound of a subject's weight for any additional days of the FMD.

9. The method of claim 1, wherein the FMD provides 3.5-5 kcal per pound of a subject's weight for day 1 of the FMD and 2.4-4 kcal per pound of a subject's weight for days 2 to 4 of the FMD.

10. The method of claim 1, wherein the FMD provides less than 25 g of sugar, less than 23 g of proteins, 16-25 grams of monounsaturated fats, 4.8-8 g of polyunsaturated fats and 1-10 g of saturated fats on day 1 of the FMD.

11. The method of claim 10, wherein the FMD provides less 16 g of sugars, less than 15 g of protein, 8-12 g of monounsaturated fats, 2-4 g of polyunsaturated fats and 1-6 grams of saturated fats on any additional days of the FMD.

12. The method of claim 1 further comprising administering a refeeding diet to the subject for a second time period.

13. The method of claim 12, wherein the second time period is at least 7 days to 6 months.

14. The method of claim 13, wherein the FMD is repeated a plurality of times at predetermined intervals.

15. The method of claim 13, wherein the FMD is administered at intervals from one week to 6 months.

16. The method of claim 1, wherein the FMD is administered from a diet package partitioned into meal portions for each day of the FMD.

17. A method for reducing senescence-positive cells in comprises:
a) identifying a subject having increased expression of p16INK4a or at risk for increased of p16INK4a; and
b) fasting by the subject for a first time period.

18. The method of claim 1, wherein the first time period is 1 to 10 days.

19. The method of claim 1, wherein the first time period is 5 days.

20. A method for reducing senescence-positive cells comprises:
administering a fasting-mimicking diet (FMD) to a subject for a first time period, the subject having been identified at increased expression of p16INK4a or at risk for increased of p16INK4a.

21. A method for prophylactically protecting against senescence-positive cells comprises:
administering a fasting-mimicking diet (FMD) to a subject for a first time period.
